(19) United States
(12) Patent Application Publication
(10) Pub. No.: US 2005/0038336 A1
(43) Pub. Date: Feb. 17, 2005

(54) Method for adapting a magnetic resonance measurement protocol to an examination subject

(76) Inventor: Ines Nimsky, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 10/846,246

(22) Filed: May 14, 2004

(30) Foreign Application Priority Data
May 16, 2003 (DE) 103 22 141.7

(51) Int. Cl. A61B 5/05

(52) U.S. Cl. 600/410

(57) ABSTRACT

In order to adapt a magnetic resonance measurement protocol to an examination subject, a magnetic resonance localization measurement is performed, Measurement data obtained In this localization measurement are evaluated. Geometric parameters characterizing the maximum physical extent of the examination subject are determined and the magnetic resonance measurement protocol is adapted to the geometric parameters. This speeds up and simplifies the execution of magnetic resonance examinatIons.

### BACKGROUND OF THE INVENTION

1. Field of the Invention

The present invention concerns a method for adapting a magnetic resonance measurement protocol to an examination subject with the aid of a magnetic resonance localization procedure.

2. Description of the Prior Art

Magnetic resonance (MR) technology is a known modality used, for example, to obtain slice images of the inside of the body of a living examination subject using magnetic resonance signals. To carry out a magnetic resonance examination, a basic field magnet produces a static, relatively homogeneous basic magnetic field. For obtaining magnetic resonance images of specifiable object or subject layers (known as “slice images”), rapidly switched gradient fields are superimposed on the basic magnetic field that are generated by gradient coils. By properly selecting the gradient fields, the slice images can be aligned in the examination subject and a spatial coding of the magnetic resonance signals necessary for spatial resolution can be achieved. The slice direction, the readout direction and the phase coding direction generally lie perpendicular to one another.

Magnetic resonance examinations are mostly performed using “magnetic resonance measurement protocols” which control the content of imaging magnetic resonance sequences. To produce a slice image with a magnetic resonance sequence, radio-frequency transmitting antennas are used to irradiate radio- frequency pulses into the examination subject to trigger magnetic resonance signals. These magnetic resonance signals are detected by one or more radio-frequency receiving antennas. The slice images of one or a number of slices (which can be specified in terms of position and orientation) of the body region of interest of the examination subject are generated based on the received magnetic resonance signals.

The reconstruction of magnetic resonance images requires an unambiguous spatial coding of the measured data. For the spatial coding, the size of the image or measurement field (FoV =field of view) must be specified for recording the region of interest. In the normal case, the region of sensitivity of a receiving antenna is larger than the FOV, which refers to the examination subject. For a successful examination, a purposeful adaptation of the FOV to the subject size is required. The adaptation includes, for example, positioning a slice image and determination of its size as well as the number of the slice images, which usually lie parallel to one another. The adaptation must take into account the phase coding since otherwise ambiguous signal codings occur that lead to artifacts in the reconstructed MR images.

The best possible adaptation of the FOV and phase coding to the region of interest is desirable since the FOV and spatial resolution are interrelated. Particularly in the case of skewed planes, i.e., magnetic resonance imaging of slices whose normal orientation does not agree with the orthogonal spatial direction of the basic field or the direction of the main body axis, it is difficult to estimate the required adaptation of the FOV for which no (or only a specifiable degree of) artifacts occur in the magnetic resonance image.

Accordingly, usually a multidimensional magnetic resonance localization measurement is performed to obtain, for example, magnetic resonance images with a coarse resolution in the plane of the slice images to be subsequently obtained as well as in two planes that are oriented perpendicularly to this plane and to one another. Based on this magnetic resonance localization measurement, the magnetic resonance measurement protocol is adapted manually by an operator of the MR equipment who is carrying out the examination by entering the position and dimensions of the FOV as well as the number of slices. This has to be performed anew for each measurement protocol and requires a great deal of experience and time.

As a starting point for the adaptation, normally in a magnetic resonance measurement protocol a slice count having a fixed setting is predefined . This corresponds to an average body volume of an average patient. The volume or the girth can vary widely from patient to patient and the magnetic resonance operator must increase or decrease the slice count based on the localization measurement for extremely obese or extremely thin patients corresponding to the patient volume or patient girth. For inexperienced personnel, this occupies valuable measurement time and the workflow is disrupted drastically.

Moreover, the phase coding and readout directions can be manually exchanged in order to optimize the measurement protocol and minimize artifacts. The phase coding direction is chosen in the direction of the shortest axis of the two-dimensional measurement field. Occasionally, additional saturation pulses are switched within the excitation pulse sequence in order to decrease undesired signal contributions from what is known as a “saturation region”, e.g., in the form of artifacts in the MR image. All of these measures are manually entered by the operator of the equipment and require a significant amount of experience.

### SUMMARY OF THE INVENTION

An object of the present invention is to simplify and speed up the execution of magnetic resonance measurement protocols.

This object is achieved according to the present invention by a method for adapting a magnetic resonance measurement protocol to an examination subject with the aid of measurement data from a multidimensional magnetic resonance localization measurement of the examination subject, wherein the magnetic resonance localization measurement is first executed and the associated measurement data are obtained, then the measurement data are evaluated and geometric parameters for characterizing the maximum physical extent of the examination subject in each measured dimension are determined and the magnetic resonance measurement protocol are adapted to the geometric parameters.

In an magnetic resonance measurement protocol, all of the settings, parameters and values are combined that define a magnetic resonance measurement for an examination which can be started by calling up the magnetic resonance measurement protocol. A magnetic resonance measurement protocol can contain, for example, the FOV that characterizes the region of interest of the examination subject that is to be imaged in the magnetic resonance measurement. The FOV is determined by the size of the slice, i.e., the length, width and thickness of a region underlying a slice image, and by the number of slices lying to parallel to one another. Moreover, in the magnetic resonance measurement protocol the course of the phase in the phase coding direction and the phase coding direction itself are determined.

The magnetic resonance measurement is conducted using MR equipment. The examination subject is, for example, a patient to be examined or a body part of the patient to be examined. The patient is brought for examination into the imaging region (volume) of the MR equipment. By means of the magnetic resonance localization measurement, the position of the examination subject is determined in the imaging region of the MR equipment, which usually is in the region of the most homogeneous basic magnetic field.

In order to perform the magnetic resonance localization measurement quickly, it is advantageous for it to have a low resolution, e.g., in comparison with the more precise magnetic resonance imaging to be performed subsequently for the diagnostic examination. Moreover, it is advantageous for the magnetic resonance localization measurement to include a number of slice images in a plane, and to obtain MR data for a number of planes that are coordinated in terms of their orientation with respect to one another in the magnetic resonance measurement protocol.

In this manner, in a number of dimensions (two dimensions for a slice measurement or three dimensions for a measurement of a series of parallel slices for 3D measurement), the examination subject can be detected in terms of position in the MR equipment.

The measurement data of the magnetic resonance localization measurement correspond to the usual signal intensity distributions of magnetic resonance measurements, only with the resolution being lower and thus the pixel structure of the magnetic resonance localization measurement being more coarse, i.e., the measured intensity of an individual pixel represents a larger volume in the subject.

Subsequently, the measurement data are evaluated automatically and geometric parameters are determined. These parameters characterize in at least one of the measured dimensions the physical extent of the examination subject. Subsequently, the magnetic resonance measurement protocol is adapted to the geometric parameters, for example, the FOV and the phase coding are adapted.

A benefit of the method according to the invention is that the magnetic resonance measurement protocol is automatically adapted to the dimensions that vary from patient to patient of the body parts to be examined. No manual input is required for this adaptation so that, for example, a magnetic resonance measurement can be started by means of the magnetic resonance measurement protocol automatically after performing the localization measurement. Under certain circumstances, it is advantageous after the automatic adaptation to offer the operator the possibility of making a check and possibly a correction.

A further benefit is that the adaptation of the magnetic resonance measurement protocol takes place faster than a manual adaptation and as a result the workflow of the magnetic resonance examination is considerably simplified and speeded up. This leads to a shortened time on average for the patient in the MR equipment.

In a further embodiment of the method, in the evaluation of the measurement data, at least one limit point of the measurement data in one dimension is determined which divides the magnetic resonance localization measurement in that dimension into two regions, of which one has essentially no measurement data points with a signal contribution from the examination subject and the other has essentially all measurement data points which have a signal contribution from the examination subject. The evaluation of the measurement data can take place using the region having signal contributions of the measurement data. For example, over a number of lines of the measurement data, the signal contributions can be accumulated and the accumulated signal evaluated. A benefit of this embodiment is that the limit point is determined through which the edge of the examination region extends in one dimension and which can be identified directly as a geometric parameter in the magnetic resonance measurement protocol.

In a further embodiment of the method, two limit points are determined in a dimension and the spacing therebetween is determined as an object-dependent (subject-dependent) parameter. An examination region in the magnetic resonance measurement protocol then can be set, for example, with a limit point, or with a limit coordinate associated with it, and the spacing between two limit points determined in this dimension.

In a further embodiment, the setting of the examination region in the magnetic resonance measurement protocol takes place with the aid of a subject-dependent isocenter that is computed using the limit points. This has the benefit that the isocenter of the magnetic resonance measurement protocol is adapted to the position of the examination subject in the magnetic resonance equipment and it is thus possible to start from the subject-dependent isocenter for image processing.

In another embodiment of the method, the phase coding extends beyond the limit points determined in the dimension corresponding to the phase coding direction in order to prevent aliased signal contributions. This has the benefit that aliasing effects are automatically prevented in the magnetic resonance imaging without the operator having to set the phase coding beforehand.

In a further embodiment, saturation regions are defined and positioned with the aid of the limit points in order to prevent interference signal contributions. This has the benefit that, in the magnetic resonance measurement protocol saturation regions are automatically defined which are adapted in terms of their position to the limit points, and thus also to the examination region. This simplifies and speeds up the usage of saturation regions in magnetic resonance measurement protocols.

In a further embodiment, with the aid of the geometric parameters, the number of slice images to be obtained in the magnetic resonance measurement protocol is computed incorporating an adjustable slice thickness. This can take place particularly with the use of the spacing between two limit points and the examination region defined in this manner.

In another embodiment, the determined parameters are transferred when calling up a further magnetic resonance measurement protocol. This has the benefit that the magnetic resonance localization measurement has to be performed only once for a number of magnetic resonance measurements having respectively different magnetic resonance measurement protocols. Time is saved accordingly.

### DESCRIPTION OF THE DRAWINGS

FIG. 1 is a flowchart for illustrating the inventive method.

FIG. 2 shows an exemplary magnetic resonance localization measurement with three magnetic resonance images in three orthogonal directions, obtained in accordance with the inventive method.

FIG. 3 is an illustration of an exemplary procedure according to the inventive method based on the example of the magnetic resonance localization measurement from FIG. 1.

FIG. 4 illustrates an exemplary procedure for determining limit points in the magnetic resonance localization measurement from FIG. 1.

FIG. 5 illustrates an adapted examination region using the magnetic resonance localization measurement from FIG. 1.

FIG. 6: is an illustration explaining the computation of an isocenter and a number of slice images to be performed and the usage of saturation regions, based on the magnetic resonance localization measurement from FIG. 1.

FIG. 7 is a table of possible geometric parameters, obtained in accordance with the inventive method, of a magnetic resonance measurement protocol.

### DESCRIPTION OF THE PREFERRED EMBODIMENTS

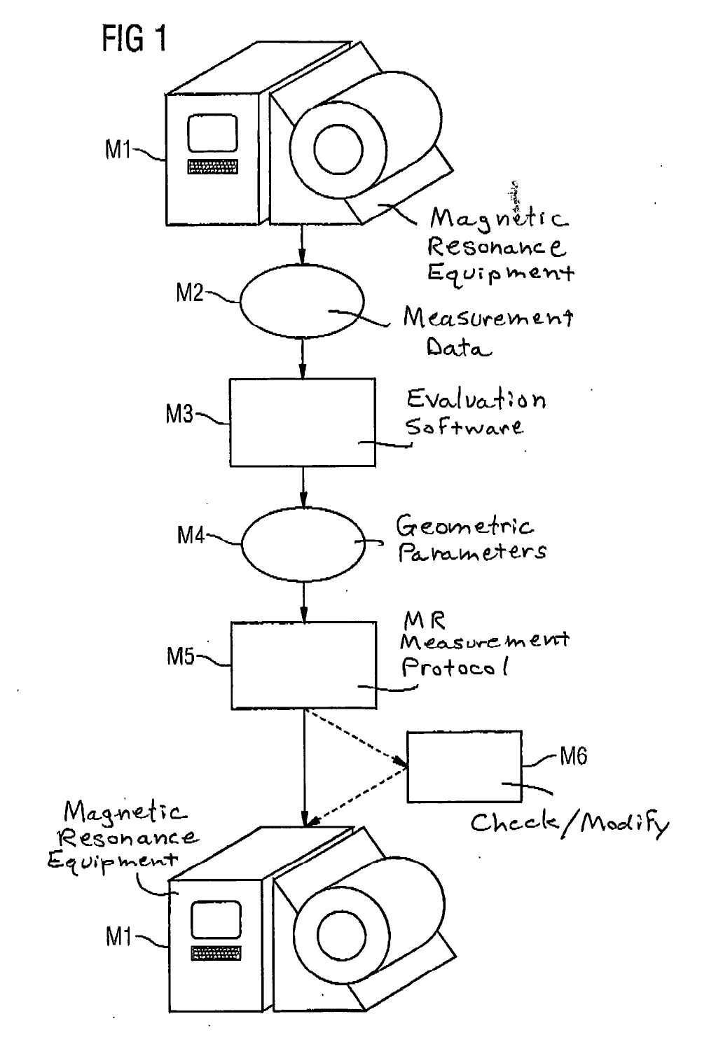

FIG. 1 is a flowchart for the inventive method. Magnetic resonance equipment M1 is used to examine a patient. Possible application areas of the method include examinations of the abdomen, shoulder, knee, heart, spinal column, and head, particularly of a child. After accommodating and introducing the patient into an imaging region of magnetic resonance equipment M1, a multidimensional magnetic resonance localization measurement is performed. Measurement data M2 are obtained which are evaluated using software M3 that can be integrated into the evaluation and control software of magnetic resonance equipment M1. Geometric parameters M4 are determined which characterize the maximum physical extent of the examination subject in each of the measured dimensions. A magnetic resonance measurement protocol M5 is adapted to the geometric parameters M4. With the adapted magnetic resonance measurement protocol M5, the diagnostic examination is performed, it being possible in a control step M6 to check and modify the magnetic resonance measurement protocol M5.

The method is explained hereafter using the example of an abdominal examination that is based on a magnetic resonance localization measurement. Preferably, the magnetic resonance images obtained in the magnetic resonance localization measurement are adapted, in terms of their orientation, to the subsequent MR measurement of the magnetic resonance protocol.

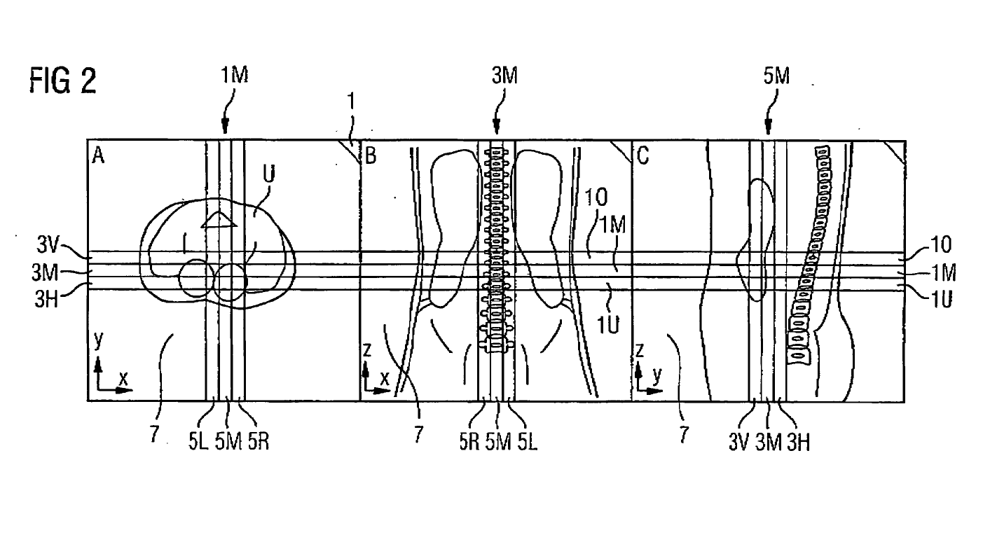

FIG. 2 shows schematically a result of a magnetic resonance localization measurement in three dimensions with MR images, which were measured with a low resolution of 256×256 pixels in three orthogonal profile directions. In each slice plane associated with a profile direction, three magnetic resonance images lying parallel to one another are measured, in each case the middle MR image in FIG. 1 being represented in an exemplary three-windowed screen display. Thus, window A shows a transverse slice image 1M, window B a coronary slice image 3M and window C a sagittal slice image 5M of the abdomen.

Additionally in each MR image, the orientation of the two other MR images extending orthogonally to the shown slice plane is indicated. For example, we see in window A a number of lines can be seen extending in conformity with the X-Y-Z coordinate system, in the X direction. These lines designate a front coronary slice image 3V, the middle coronary slice image 3M and a rear slice image 3H. Perpendicular lines in the Y direction mark the position of a left sagittal slice image 5L, the middle sagittal slice image 5M, and a right sagiftal slice image 5R. The orientation of the magnetic resonance images is shown correspondingly in the windows B and C. In the window C also an upper transverse slice image 1O, the middle transverse slice image 1M and a lower transverse slice image 1U can be seen.

What is measured and represented is essentially the entire imaging region of the magnetic resonance equipment that is determined by the used receiving antennas. In the schematically shown slice images 1M, 3M, 5M, an examination subject U can be recognized.

In one possible way of representing an MR image, regions with a high proton density, e.g., water or fatty tissue, which emit a strong magnetic resonance signal and thus have a high signal intensity, are shown as lighter images. Correspondingly, the examination subject U has in the inside different grey scales depending on the proton concentration. A space 7 surrounding the examination subject U produces essentially no signal and is represented normally in a magnetic resonance image in black. For clarity, in FIG. 2 only structures in the examination subject U are reproduced schematically with lines. Grey shades for representing the signal level in order to make clear, for example, the signal-free space 7 are not shown.

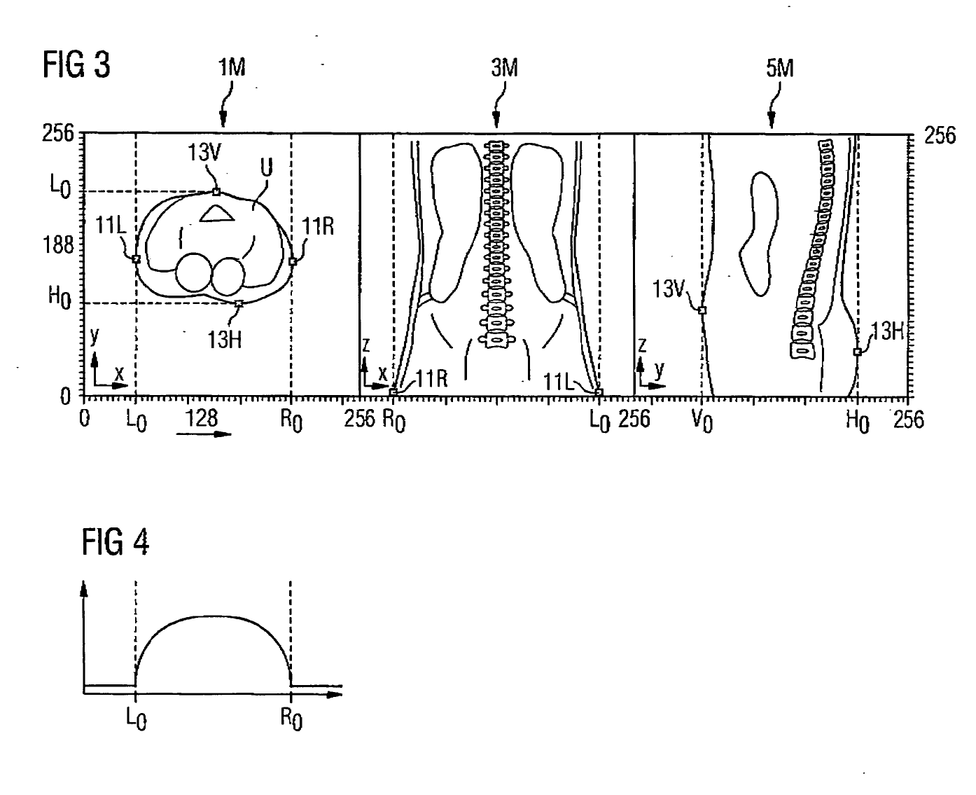

FIG. 3 illustrates the function of limit points in the method based on the magnetic resonance localization measurement from FIG. 2. The measurement data from the magnetic resonance localization measurement are used to determine the limit points. For clarity, the lines for designating the orthogonal MR images are not shown any more. Instead, the pixel structure of the MR images 1M, 3M, 5M (which include 256×256 pixels in each case) is indicated at the image edges.

The limit points 11L, 11R, 13V, 13H are recognizable, and correspond in each case to one pixel that indicate the maximum extension of the examination subject U in one dimension. The limit coordinates L0, R0, V0, H0 of the limit points 11L, . . . 13H in the respective dimension are marked at the edge of the image.

For example, one of the limit points 11L, 11R, 13V, 13H can be determined based on the distribution of the slice images 1M, 3M, 5M into regions with and without a signal. For this purpose, a perpendicular line is drawn which extends through the limit point 11L and correspondingly through the limit coordinate L0. Between the line and the left edge of the MR image, there is not another pixel that has an intensity contribution, i.e., no part of the examination subject U is located in this part of the sensitive region. This means the entire examination subject U is located on the right side of the line. Corresponding lines are drawn through the limit point 11R in the slice image 3M as well as through the limit point 13V in the slice image 5M.

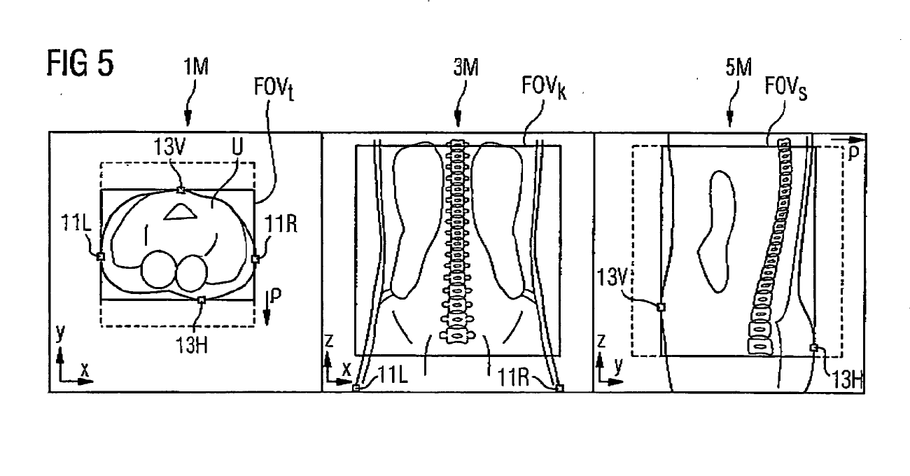

FIG. 4 shows an exemplary procedure for determining the limit points 11L, 11R. For this purpose, the transverse slice image 1M was integrated in terms of its intensity in the Y direction. The intensity integrated over the spatial coordinate X is shown in FIG. 4. Additionally, a line through the point 11L corresponding to FIG. 3 is indicated. To the left of the line, i.e., for pixels with X coordinates less than L0, almost no intensity is accumulated. Between the pixels L0 and R0, the examination subject is located and accordingly these pixels exhibit a high-accumulated intensity. In pixels with an X coordinate greater than R0, integration is performed again over an area free of the examination subject so that there again a negligible intensity signal is present. Using a simple algorithm, it can now be determined where the examination subject U begins or ends, i.e., analogously the limit coordinates L₀, R₀, V₀, H₀ can be determined. Under certain circumstances, it is advantageous to take into account a background signal.

Based on the limit points 11L, . . . 13H, which themselves are already geometric parameters for characterizing the maximum extension of the examination subject U, further parameters can be determined such as the spacing between two of the limit points 11L, . . . 13H in one dimension as well as the center point between two limit points.

After the evaluation of the measurement data of the magnetic resonance localization measurement, the examination region of the measurement protocol can be adapted. FIG. 5 illustrates this schematically. In the transverse slice image 1M, a transverse examination region FOV_{T} is shown using a rectangular with sides extending through the limit points 11L, 11R, 13V and 13H.

To avoid artifacts, the phase coding is extended in a direction p indicated with an arrow by in each case several percent beyond the limit point 13V, 13H. This is particularly important, for example, when “zooming” the examination regions FOV_{T}. This can be desirable, for example, if an examination region is selected that is smaller than the examination region proposed through the limit points, in order to suppress undesired signal contributions from adjacent areas. With the aid of the geometric parameters, the optimum phase coding direction can be selected and the extension of the phase coding can be automatically adapted.

In the coronary slice image 3M, a further special case has occurred. Since no limit point can be determined based on the abdomen examination in the Z direction, in the Z direction the entire sensitive region is selected except for a narrow edge as the coronary examination region FOV_{K}. The lateral edges extend in their extension through the limit points 11R and 11L.

The sagittal slice image 5M is likewise a special case in the Z direction; however, the limit points 13V, 13H lie on the lines of the rectangle which indicates a sagittal examination region FOV_{S}. Here as well, the extension of the phase coding direction in the Y direction is shown with a dashed line.

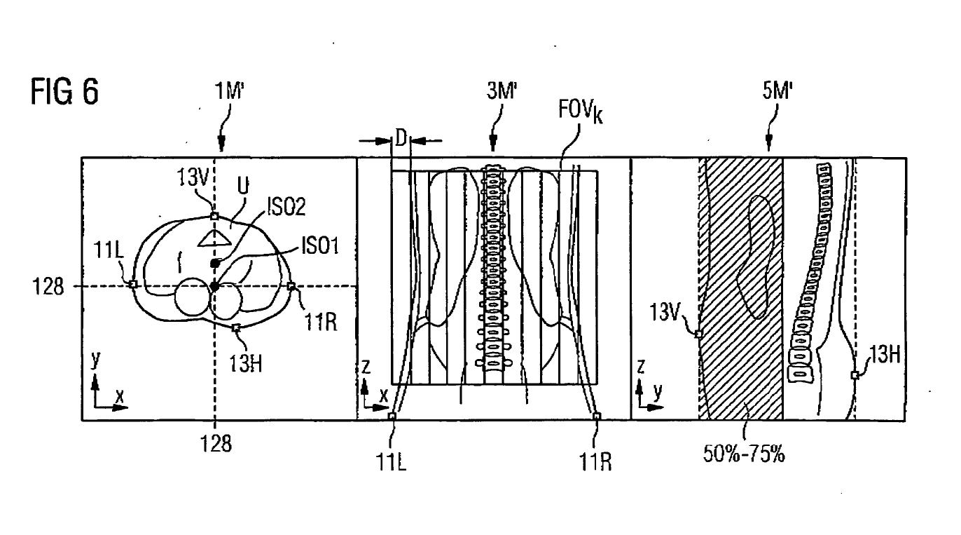

FIG. 6 illustrates further aspects of the method. For example, in the transverse slice image 1M′, the isocenter ISO1, i.e., the center of the sensitive region, is shown. Additionally, the isocenter ISO2 is shown that indicates the center of the subject based on the examination region FOV determined with the aid of the limit points 11L, . . . 13H. Since the examination subject cannot always be ideally positioned in the magnetic resonance equipment, the two isocenters ISO1, ISO2 do not coincide in most cases.

In the slice image 3M′, it is illustrated that, using the spacings of the limit points 11L, 11R and with a preset slice thickness D, for example, of 10 mm, the number of transverse images to be obtained can be computed and marked in the magnetic resonance localization measurement. The scope of the examination region FOV_{K} in the X direction is adapted to an integral multiple of the slice thickness D.

The slice image 5M′ illustrates the use of saturation regions based on the parameters determined from the localization measurement data. For example, in case of a sagittal slice image for a spinal column examination, the front region of the abdomen could be saturated by a saturation pulse in its signal contribution.

In order to suppress, for example, interference due to heart and respiratory activity in a spinal column examination in a presetting starting from the limit point 13V a saturation region S from 50 to 75% of the spacing between the limit points 13V and 13H can be proposed in an automated manner in the magnetic resonance measurement protocol. In a shoulder examination, by means of a saturation region that is oriented and situated in an automated manner, the imaging region of the opposing shoulders can be saturated in order to suppress artifacts in the imaging.

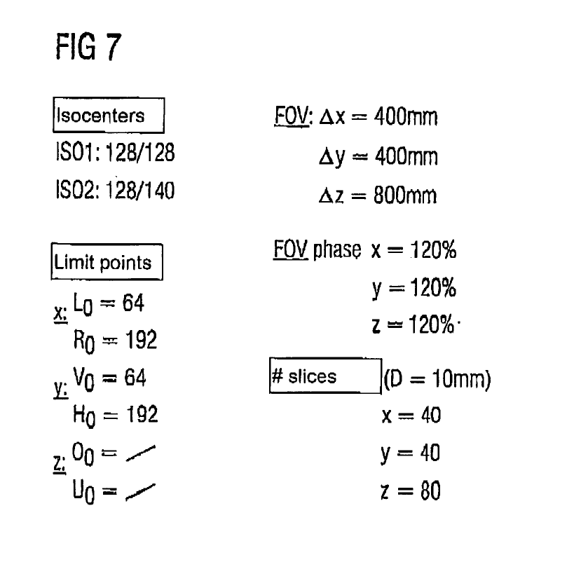

FIG. 7 shows a table of exemplary parameters that can be determined using method and implemented in a magnetic resonance measurement protocol. As an example, the table contains the positions of the isocenters ISO1 and ISO2 that characterize the center of the imaging region and the center of the examination region, respectively. Moreover, in the three dimensions, the coordinates L₀, R₀, V₀, H₀, O₀, U₀ are indicated as well as the examination regions FOV_{T}, FOV_{K}, FOV_{S} determined using the limit points 11L, . . . 13H are indicated using the widths ΔX, ΔY, ΔZ. The quantity underlying the phase coding is proposed in the X, Y, and Z directions as a percentage. Additionally, the number of slices in the different dimensions can be indicated based on the determined geometric parameters.

With the localization measurement, the body region under examination is measured in three slice planes. The measurement values obtained are evaluated to produce geometric parameters and are available as information for adapting the measurement protocol. This takes place in an automated manner and is presented to the operator as a patient-specific proposal, such as in a popup menu with a table from FIG. 7. The operator can accept the proposal, reject it or further process it manually.

The geometric parameters can be indicated, for example, as pixels of the magnetic resonance localization measurement, as pixels of the measurement of the measurement protocol or in mm quantity units. They can be stored after executing a measurement protocol and used in a subsequent measurement protocol. This can also be adapted in an automated manner and the associated magnetic resonance measurement started in an automated manner. An interim step for checking or adapting the magnetic resonance measurement protocol by the operator can be implemented therebetween.

Although modifications and changes may be suggested by those skilled in the art, it is the intention of the inventor to embody within the patent warranted hereon all changes and modifications as reasonably and properly come within the scope of her contribution to the art.

1. A method for adapting a magnetic resonance measurement protocol to an examination subject comprising the steps of:
conducting a multi-dimensional magnetic resonance localization measurement of a subject for obtaining measurement data;
evaluating said measurement data to determine geometric parameters characterizing a maximum physical extent of the subject in each dimension; and
adapting a magnetic resonance measurement protocol. to be executed on the subject, to said geometric parameters.

2. A method as claimed in claim 1 wherein said magnetic resonance measurement protocol will produce diagnostic data having a resolution associated therewith, and wherein the step of executing said magnetic resonance localization measurement comprises executing said magnetic resonance localization measurement for acquiring measurement data having a resolution lower than said resolution of said data to be acquired in said magnetic resonance measurement protocol.

3. A method as claimed in claim 1 wherein said magnetic resonance measurement protocol defines a plurality of slice images of the subject, and comprising executing said magnetic resonance localization measurement for obtaining said measurement data in said plurality of slice images, and adapting an orientation of the plurality of slice images relative to each other dependent on said geometric parameters,

4. A method as claimed in claim 1 wherein said measurement data from said magnetic resonance localization measurement have a signal distribution, and comprising evaluating the signal distribution of the measurement data for determining said geometric parameters.

5. A method as claimed in claim 1 wherein the step of evaluating said measurement data comprises, in one of said dimensions, determining a limit point dividing said measurement data in said one of said dimensions into a first region containing substantially no measurement data with a signal contribution from the subject, and a second region containing substantially all measurement data having a signal contribution from the subject.

6. A method as claimed in claim 5 comprising for said limit point, determining a limit coordinate relative to said subject as a subject-dependent parameter.

7. A method as claimed in claim 6 wherein said limit point is a first limit point, and comprising determining a second limit point in said one dimension and determining said subject-based parameter as a spacing between said first and second limit points.

8. A method as claimed in claim 7 comprising determining a limit coordinate for said second limit point, and defining an edge of an examination region in said magnetic resonance measurement protocol dependent on the respective limit coordinates of said first and second limit points.

9. A method as claimed in claim 8 comprising setting said examination region in said magnetic resonance measurement protocol in said one dimension.

10. A method as claimed in claim 7 comprising, from said first and second limit points. defining a subject-dependent isocenter for positioning an examination region in said magnetic resonance measurement protocol.

11. A method as claimed in claim 7 comprising defining said one dimension as a direction for phase coding in said magnetic resonance measurement protocol, and extending said phase coding in said one dimension beyond said first and second limit points for preventing aliasing signal contributions.

12. A method as claimed in claim 7 comprising defining a saturation region in said magnetic resonance measurement protocol dependent on said first and second limit points for preventing interference signal contributions.

13. A method as claimed in claim 7 comprising, in said magnetic resonance measurement protocol, determining a number of slice images to be obtained dependent on said spacing between said first and second limit points, by adjusting respective thicknesses of the slice images.

14. A method as claimed in claim I comprising saving at least one of said geometric parameters as a saved parameter, and calling said saved parameter when conducting a further magnetic resonance measurement protocol.
